(19) United States
(12) Patent Application Publication
(10) Pub. No.: US 2026/0042052 A1
(43) Pub. Date: Feb. 12, 2026

(54) SYSTEMS AND METHODS FOR SEPARATING A GAS MIXTURE THAT INCLUDES ORGANIC AND INORGANIC COMPOUNDS

(71) Applicant: SABIC GLOBAL TECHNOLOGIES B.V., Bergen op Zoom (NL)

(72) Inventors: Ashwin Ravi Sankar, Riyadh (SA); Mansour Ibraheem Algoblan, Riyadh (SA)

(21) Appl. No.: 19/102,933

(22) PCT Filed: Aug. 16, 2023
(86) PCT No.: PCT/EP2023/072595

§ 371 Date: Feb. 11, 2025

(30) Foreign Application Priority Data
Aug. 17, 2022 (EP) 22190783.5

(51) Int. Cl. B01D 53/22; B01D 71/16; C07C 7/144

(52) U.S. Cl. CPC B01D 53/228; B01D 71/16; C07C 7/144; B01D 2256/24; B01D 2257/11; B01D 2257/504

(57) ABSTRACT

Systems and methods for separating a gas mixture of organic gaseous compounds and inorganic gaseous compounds are disclosed. The systems include a membrane unit with a membrane element that is at least partially coated with an organic liquid. The methods include using a membrane unit with the membrane element at least partially coated with an organic liquid to separate a gas mixture of organic gaseous compounds and inorganic gaseous compounds.

### CROSS REFERENCE TO RELATED APPLICATIONS

None.

### FIELD OF INVENTION

The present disclosure generally relates to systems and methods for separating a gas mixture that comprises organic gaseous compounds and inorganic gaseous compounds. More specifically, the present disclosure relates to separating a gas mixture that comprises organic gaseous compounds and inorganic gaseous compounds with a gas membrane unit adapted for such separation.

### BACKGROUND OF THE INVENTION

Typically, in plants that produce ethylene oxide and ethylene glycol, a reclaim gas stream is formed that comprises ethylene (5-30 wt. %), carbon dioxide (30-90 wt. %), argon (1-10 wt. %), methane (1-10 wt. %), and water (0.5-5 wt. %). This reclaim gas stream is usually recycled back to the ethylene oxide reactor. There is a need to purge a certain amount of the reclaim gas stream to avoid argon and ethane build-up in the feed stream to the ethylene oxide reactor. However, the purge rate is directly proportional to the amount of ethylene (feedstock) that will be lost.

One solution that has been implemented to address the loss of ethylene caused by purging is the installation of an ethylene recovery unit (ERU), which removes argon from the reclaim gas stream and recycles the ethylene back to the ethylene oxide reactor. But even with the use of an ERU, significant purging is still required, which results in some ethylene loss. Generally, an ERU is not an effective unit operation to resolve the issue of ethylene loss. Moreover, the use of ERU in the ethylene oxide/ethylene glycol production process is not energy efficient.

### BRIEF SUMMARY OF THE INVENTION

The present inventors disclose herein the use of a membrane unit for separating a gas mixture that comprises organic compounds and inorganic compounds, in an efficient manner. The present disclosure involves separating a gas mixture (such as a reclaim gas from an ethylene oxide/ethylene glycol plant) with a membrane adapted to carry out such separation by soaking the membrane in an organic liquid and then using the pre-soaked membrane to remove carbon dioxide and argon from the reclaim gas. In the ethylene oxide/ethylene glycol production process, for example, such separation can enhance process efficiency and selectivity of the ethylene-to-ethylene oxide reaction. This separation, according to the present disclosure, can eliminate or reduce the gas purge rate, leading to more ethylene recovery and savings on feedstock.

Embodiments of the disclosure include a method of separating a gas mixture comprising organic gaseous compounds and inorganic gaseous compounds, wherein the method comprises flowing the gas mixture into a membrane unit comprising a membrane element at least partially coated with an organic liquid and separating the gas mixture in the membrane unit. The method further comprises flowing a first gas stream from the membrane unit, the first gas stream comprising a lower concentration of inorganic gaseous compounds than the gas mixture.

Embodiments of the disclosure include a method of separating a gas mixture comprising ethylene, carbon dioxide, methane, and argon. The method comprises flowing the gas mixture into a membrane unit comprising a membrane element at least partially coated with propylene oxide, ethylene oxide, methanol, benzene, or combinations thereof and separating the gas mixture in the membrane unit. The method further comprises flowing a first gas stream from the membrane unit, the first gas stream comprising a lower concentration of carbon dioxide and argon than the gas mixture.

Embodiments of the disclosure include a method of separating a gas mixture comprising ethylene, carbon dioxide, methane, and argon. The method comprises flowing the gas mixture into a membrane unit comprising a membrane element that comprises per-fluoro-polymer and/or cellulose acetate, wherein the membrane element is at least partially coated with propylene oxide, ethylene oxide, methanol, benzene, or combinations thereof and separating the gas mixture in the membrane unit. Further, the method comprises flowing an inert gas into the membrane unit as a sweeping gas. Further yet, the method further comprises flowing a first gas stream from the membrane unit, the first gas stream comprising a lower concentration of carbon dioxide and argon than the gas mixture.

Embodiments of the disclosure include a system for separating a gas mixture comprising organic gaseous compounds and inorganic gaseous compounds. The system comprises a membrane unit comprising a membrane element at least partially coated with an organic liquid.

Embodiments of the disclosure include a system for separating a gas mixture comprising organic gaseous compounds and inorganic gaseous compounds. The system comprises a membrane unit comprising a membrane element that comprises per-fluoro-polymer and/or cellulose acetate that is at least partially coated with propylene oxide, ethylene oxide, methanol, benzene, or combinations thereof.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

For the purposes of this disclosure, “X, Y, and/or Z” can be construed as X only, Y only, Z only, or any combination of two or more items X, Y, and Z (e.g., XYZ, XY, XZ, YZ).

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a system for separating a gas mixture comprising organic gaseous compounds and inorganic gaseous compounds, according to embodiments of the disclosure; and

FIG. 2 shows a method for separating a gas mixture comprising organic gaseous compounds and inorganic gaseous compounds, according to embodiments of the disclosure.

### DETAILED DESCRIPTION OF THE INVENTION

According to the present disclosure, a mixture of organic gas and inorganic gas can be efficiently separated by using a membrane that is soaked in an organic liquid. For example, in an ethylene oxide/ethylene glycol plant, a reclaim gas mixture comprising ethylene, carbon dioxide, methane, and argon can be separated by flowing the gas mixture into a membrane unit comprising a membrane element that comprises per-fluoro-polymer and/or cellulose acetate, after the membrane element has been soaked in and thus at least partially coated with propylene oxide, ethylene oxide, methanol, benzene, or combinations thereof. In embodiments of the disclosure, the coating of the membrane element with propylene oxide, ethylene oxide, methanol, benzene, or combinations thereof can be implemented by methods other than, or in addition to, soaking, such as spraying or dip-coating.

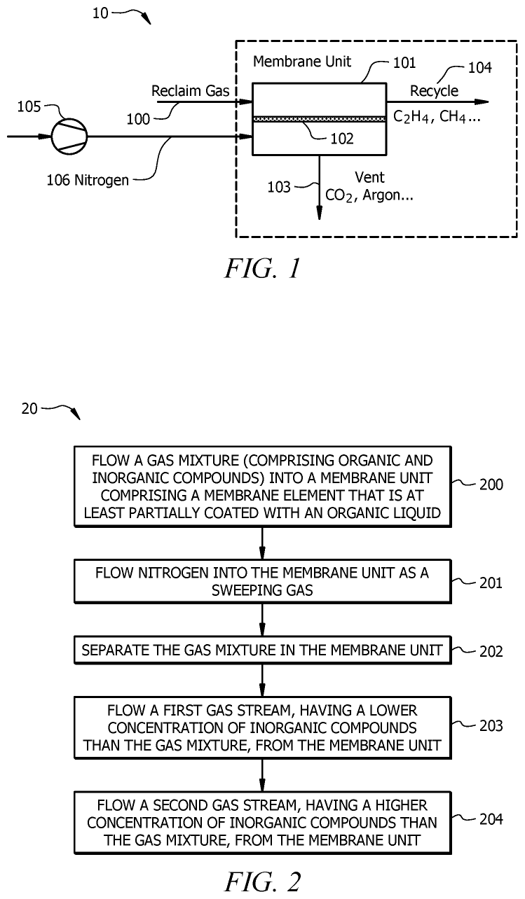

FIG. 1 shows a system 10 for separating a gas mixture comprising organic gaseous compounds and inorganic gaseous compounds, according to embodiments of the disclosure. FIG. 2 shows a method 20 for separating a gas mixture comprising organic gaseous compounds and inorganic gaseous compounds, according to embodiments of the disclosure. Method 20, in embodiments of the disclosure, is implemented by system 10.

### Systems for Separating a Gas Mixture Comprising Organic and Inorganic Compounds

According to embodiments of the disclosure, system 10 includes a membrane unit 101, which has disposed therein a membrane element 102. Membrane element 102, according to embodiments of the disclosure, comprises perfluoro-polymer or similar glassy polymeric material, ceramic or hybrid polymer matrix-ceramic material (per-fluoro-polymer and/or cellulose acetate). Further, according to embodiments of the disclosure, membrane element 102 is at least partially coated with an organic liquid such as propylene oxide, ethylene oxide, methanol, benzene, or combinations thereof. The coating of membrane element 102 with propylene oxide, ethylene oxide, methanol, benzene, or combinations thereof can be achieved by soaking membrane element 102 in propylene oxide, ethylene oxide, methanol, benzene, or combinations thereof. Alternatively, the organic liquid may be sprayed or dip-coated onto membrane element 102. In an aspect, the organic liquid is flowed through or otherwise contacts membrane element 102 for 2 to 5 hours, or 2.5 to 3 hours, and then dried or cured before carrying out a separation step with the membrane element. Alternatively, the organic liquid is sprayed onto the membrane element for 20 to 60 minutes, or 25 to 35 minutes, and then dried or cured before carrying out a separation step. In another aspect, membrane element 102 is dip-coated in the organic liquid for 10 to 30 minutes, or 15 to 20 minutes, and then dried or cured before carrying out a separation step. It has been discovered that such contact with the organic liquid forms a coating or boundary of organic liquid in or on the membrane element. This is found to help achieve accelerated and more efficient separation as described herein.

In this way, membrane unit 101 is adapted to receive and separate, for example, reclaim gas 100 (gas mixture), comprising ethylene, carbon dioxide, methane, and argon, and thereby form (1) a first gas stream 104 (recycle gas), comprising ethylene and methane, and (2) a second gas stream 103 (vent gas) comprising carbon dioxide and argon. Thus, membrane unit 101, according to embodiments of the disclosure, is adapted to separate reclaim gas 100 to produce first gas stream 104 that comprises a lower concentration of carbon dioxide and argon (and a higher concentration of ethylene and methane) than reclaim gas 100. Correspondingly, membrane unit 101 is adapted to separate reclaim gas 100 to produce second gas stream 103 that comprises a higher concentration of carbon dioxide and argon (and a lower concentration of ethylene and methane) than reclaim gas 100. In embodiments of the disclosure, system 10 comprises a compressor unit 105 adapted to flow nitrogen 106 through membrane unit 101 as a sweeping gas.

### Methods for Separating a Gas Mixture Comprising Organic and Inorganic Compounds

Method 20 may be implemented, for example, with respect to a gas mixture that is a reclaim gas stream from an ethylene oxide/ethylene glycol production plant. According to embodiments of the disclosure, method 20 includes, at block 200, and as implemented using system 10, flowing reclaim gas 100 (gas mixture) into membrane unit 101, wherein membrane unit 101 comprises membrane element 102 that is at least partially coated with an organic liquid, such as propylene oxide, ethylene oxide, methanol, benzene, or combinations thereof. According to embodiments of the disclosure, membrane element 102 comprises per-fluoro-polymer and/or cellulose acetate. The coating of membrane element 102, according to embodiments of the disclosure, can include soaking membrane element 102 in the organic liquid (e.g., propylene oxide, ethylene oxide, methanol, benzene, or combinations thereof) for 2-5 hours before the flowing of reclaim gas 100 into membrane unit 101.

According to embodiments of the disclosure, reclaim gas 100 comprises ethylene, carbon dioxide, methane, and argon. Reclaim gas 100, in some ethylene oxide/ethylene glycol production plants, in which this disclosure can be applied, can comprise 5 to 30 wt. % ethylene, 30 to 90 wt. % carbon dioxide, 1 to 10 wt. % argon, 1 to 10 wt. % methane, and 0.5 to 5 wt. % water.

Method 20, according to embodiments of the disclosure, includes, at block 201, flowing nitrogen 106 at 20 to 30% of mass flow into membrane unit 101 as a sweeping gas. In this way, according to embodiments of the disclosure, the separation at block 200 can be achieved using a combination of sweeping gas membrane separation, partial pressure difference, as well as pore size filtration based on molecular size. Nitrogen can used as a sweeping gas to further push the mass transfer, creating the partial pressure difference between the feed and the permeate (second gas stream 103) side. On the retentate (first gas stream 104) side, the ethylene can be recovered and recycled back to the ethylene oxide reactor along with the methane (CH₄), which serves as a ballast gas for heat transfer control. Steam or nitrogen may also be used as ballast gas.

Method 20, according to embodiments of the disclosure, further includes, at block 202, separating reclaim gas 100 in membrane unit 101, by membrane element 102; and, at block 203, flowing first gas stream 104 from membrane unit 101. At block 202, carbon dioxide and argon, which have lower molecular size than ethylene, can permeate membrane element 102 more than hydrocarbons and ethylene, and so the ethylene and other hydrocarbons will remain on the high pressure side (i.e., the retentate side), according to embodiments of the disclosure. The operating conditions in membrane unit 101, in embodiments of the disclosure, comprise a temperature of 80 to 200° C., including 80 to 90° C., 90 to 100° C., 100 to 110° C., 110 to 120° C., 120 to 130° C., 130 to 140° C., 140 to 150° C., 150 to 160° C., 160 to 170° C., 170 to 180° C., 180 to 190° C., and 190 to 200° C.; a pressure of 10 to 50 bar, including 10 to 15 bar, 15 to 20 bar, 20 to 25 bar, 25 to 30 bar, 30 to 35 bar, 35 to 40 bar, 40 to 45 bar, and 45 to 50 bar; and a flow rate of 200 to 30000 kg/hour, including 200 to 2000, kg/hour, 2000 to 4000 kg/hour, 4000 to 6000 kg/hour, 6000 to 8000 kg/hour, 8000 to 10000 kg/hour, 10000 to 12000 kg/hour, 12000 to 14000 kg/hour, 14000 to 16000 kg/hour, 16000 to 18000 kg/hour, 18000 to 20000 kg/hour, 20000 to 22000 kg/hour, 22000 to 24000 kg/hour, 24000 to 26000 kg/hour, 26000 to 28000 kg/hour, and 28000 to 30000 kg/hour.

According to embodiments of the disclosure, as a result of the separating at block 200, first gas stream 104 has a lower concentration of inorganic gaseous compounds, carbon dioxide and argon (and a higher concentration of ethylene and methane) than reclaim gas 100. First gas stream 104 comprises, in embodiments of the disclosure, 10 to 45 wt. % ethylene, 20 to 50 wt. % carbon dioxide, 0.5 to 8 wt. % argon, 5 to 20 wt. % methane, and 0.01 to 3 wt. % water.

At block 204, according to embodiments of the disclosure, method 20 includes flowing second gas stream 103 from membrane unit 101. In embodiments of the disclosure, second gas stream 103 comprises a higher concentration of carbon dioxide and argon (and a lower concentration of ethylene and methane) than reclaim gas 100. Second gas stream 103, in embodiments of the disclosure, comprises 0.1 to 4 wt. % ethylene, 70 to 98 wt. % carbon dioxide, 1 to 20 wt. % argon, 0.4 to 8 wt. % methane, and 0.1 to 1 wt. % water. In embodiments of the disclosure, 85 to 87 wt. % carbon dioxide and 30 to 60 wt. % argon can be removed from reclaim gas 100 and 90 to 93 wt. % of ethylene can be recovered in first gas stream 104.

According to embodiments of the disclosure, the carbon dioxide removal rate without soaking is in the range of 40% to 60%, alternatively in the range of 42% to 58%, alternatively in the range of 44% to 58%, alternatively in the range of 45% to 58%, alternatively in the range of 48% to 58%, whereas with soaking in feed/PO or EO or benzene or methanol or combinations thereof the carbon dioxide removal rate is in the range of 70% to 90%, alternatively in the range of 70% to 90%, alternatively in the range of 72% to 90%, alternatively in the range of 75% to 88% whereas in combination with soaking and sweeping gas the carbon dioxide removal rate is in the range of 75% to 98%, alternatively in the range of 75% to 99%, alternatively in the range of 78% to 99%, alternatively in the range of 80% to 99%, alternatively in the range of 80% to 97%.

Although embodiments of the present disclosure have been described with reference to blocks of FIG. 2 it should be appreciated that operation of the present disclosure is not limited to the particular blocks and/or the particular order of the blocks illustrated in FIG. 2. Accordingly, embodiments of the disclosure may provide functionality as described herein using various blocks in a sequence different than that of FIG. 2.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present invention, specific examples are included below. The examples are for illustrative purposes only and are not intended to limit the disclosure. Those of ordinary skill in the art will readily recognize parameters that can be changed or modified to yield essentially the same results.

### EXAMPLE

### Lab Scale Experiment-Membrane Stability

Experiments were conducted at a lab scale to test the stability of membranes when exposed to hydrocarbon streams including ethylene oxide. As part of the test, the membranes were soaked for 2-4 hours to measure increase in separation efficiency.

Two types of membranes were soaked in propylene oxide and their respective separation performance was measured before and after soaking. The types of membranes used in the experiment were: (1) Per-fluoro-polymer and (2) cellulose acetate.

The results from the tests are shown in Table 1 below.

TABLE 1

| | Before Soaking in | | Stability Test - After Soaking | |
|---|---|---|---|---|
| | Propylene Oxide | | in Propylene Oxide | |
| | Membrane A | Membrane B | Membrane A | Membrane B |
| | (Perfluoropolymer) | (cellulose acetate) | (Perfluoropolymer) | (cellulose acetate) |
| Pure-Gas Test | Permeance (GPU) | Permeance (GPU) | Permeance (GPU) | Permeance (GPU) |
| N₂ | 81.53 | 2.07 | 71.59 | Not Stable in PO |
| O₂ | — | 10.87 | — | |
| CO₂ | 587.04 | 67.48 | 533.68 | |
| C₂H₄ | 18.94 | 2.67 | 16.77 | |
| Ar | 127.62 | 5.06 | — | |
| CH₄ | 37.87 | 1.96 | 34.94 | |
| | Selectivity | Selectivity | Selectivity | Selectivity |
| CO₂/C₂H₄ | 31 | 25.3 | 31.8 | |
| CO₂/CH₄ | 15.5 | 34.5 | 15.3 | |
| Ar/C₂H₄ | 6.7 | 1.9 | — | |
| Ar/CH₄ | 3.4 | 2.6 | — | |

* 1 GPU = 10⁻⁶ cm³/(cm² · s · cmHg)
# Stability test: soak in propylene oxide and then dried in vacuum

From the results shown in Table 1, the perfluoropolymer is stable and efficient in the targeted stream to be separated.

TABLE 2

| | Before Soaking in | | Stability Test - After | | Stability Test - After | |
|---|---|---|---|---|---|---|
| | Benzene or Methanol | | Soaking in Benzene | | Soaking in Methanol | |
| | Membrane A | Membrane B | Membrane A | Membrane B | Membrane A | Membrane B |
| Pure Gas | (Perfluoropolymer) | (cellulose acetate) | (Perfluoropolymer) | (cellulose acetate) | (Perfluoropolymer) | (cellulose acetate) |
| Test | Permeance (GPU) | Permeance (GPU) | Permeance (GPU) | Permeance (GPU) | Permeance (GPU) | Permeance (GPU) |
| N₂ | 81.56 | 2.17 | 70.52 | Semi-stable in | 69.95 | Semi-stable |
| | | | | Benzene | | in Methanol |
| O₂ | — | 9.78 | — | | — | |
| CO₂ | 585.14 | 65.48 | 529.67 | 43.2 | 531.26 | 42.5 |
| C₂H₄ | 19.14 | 1.76 | 18.82 | 0.4 | 19.13 | 0.7 |
| Ar | 129.62 | 4.16 | 130.15 | 2.67 | 131.05 | 3.5 |
| CH₄ | 36.74 | 2.02 | 35.24 | 1.87 | 36.65 | 1.98 |
| | Selectivity | Selectivity | Selectivity | Selectivity | Selectivity | |
| CO₂/C₂H₄ | 31.5 | 22.3 | 31.2 | 15.6 | 31.3 | 14.2 |
| CO₂/CH₄ | 16.5 | 31.5 | 16.1 | 24.5 | 15.2 | 12.5 |
| Ar/C₂H₄ | 7.7 | 2.1 | 7.8 | 1.2 | 7.7 | 1.1 |
| Ar/CH₄ | 3.2 | 2.5 | 3.5 | 1.7 | 3.4 | 0.8 |

* 1 GPU = 10⁻⁶ cm³/(cm² · s · cmHg)
# Stability test: soak in Benzene, Methanol and then dried in vacuum

From the results shown in Table 2, the perfluoropolymer is stable and efficient in the targeted stream to be separated.

### Simulations of Membrane Separation Process

5 Simulations were conducted for a pilot scale test using Aspen Custom Modeler, JMP (DOE) and a plant scenario case using Aspen v.9. The results are shown in Table 3 and Table 4, respectively.

TABLE 3
DOE Predicted Data - Membrane A (Perfluoropolymer)

| Temperature | Pressure | CO₂ Concentration | Permeance (GPU) | Selectivity |
|---|---|---|---|---|
| (° C.) | (bar) | (wt.) | CO₂ | (CO₂/C₂H₄) |
| 95 | 20 | 30 | 556 | 28.5 |
| 115 | 20 | 35 | 589 | 29.2 |
| 130 | 25 | 40 | 601 | 30.2 |
| 135 | 25 | 60 | 620 | 32.3 |
| 150 | 30 | 80 | 627 | 33.5 |

```
Calculation of permeate and retentate molar flow rates at i th stage
  do k=1,7,1
    P|(k,i)=Perm(k)*100*3600*(PF*yr(k,i)−PP*yp(k,i))
    P|A(k,i)=theta(i)*F(i)yp(k,i)
    R(k,i)=F(i)*yf(k,i)−P|A(k,i)
  end do
```

TABLE 4
PLANT ASSESSMENT

| Stream Name | Feed | Retentate | Permeate | Purge | |
|---|---|---|---|---|---|
| Pressure, bar | 20 | 18.5 | 1.3 | 17 | Membrane system |
| Temperature, ° C. | 110 | 34 | 44 | 45 | removes 6.5 t/h |
| Mass Flow, kg/h | 8960 | 2135 | 6746 | 605 | CO₂ from reclaim |
| Composition, wt. % | | | | | gas - 0.1% selectivity |
| Ethylene | 9.3 | 36.3 | 0.86 | 27.15 | increase saves 500 t/y |
| Oxygen | 0.7 | 1.04 | 0.6 | 9.91 | C₂H₄/500 KTA EOE |
| Carbon Dioxide | 83 | 43.93 | 96.33 | 13.63 | Purge gas flow rate |
| Water | 1 | 0.01 | 0.16 | 0.23 | to be significantly |
| Nitrogen | 0.6 | 2.05 | 0.15 | 0.33 | reduced, potential |
| Argon | 2.2 | 4.9 | 1.37 | 18.1 | C₂H₄ saving 850 t/h |
| Methane | 3.2 | 11.77 | 0.53 | 27.09 | |
| Component Mass Flow, kg/hr | | | | | |
| Ethylene | 833 | 775 | 58 | 164 | |
| Oxygen | 63 | 22 | 40 | 60 | |
| Carbon Dioxide | 7437 | 938 | 6498 | 82 | |
| Water | 90 | 0 | 11 | 1 | |
| Nitrogen | 54 | 44 | 10 | 2 | |
| Argon | 197 | 105 | 92 | 110 | |
| Methane | 287 | 251 | 36 | 164 | |

### Experimental Data on the Effects of Soaking & Sweeping Gas on Separation Performance

Tests were conducted to determine the effects of soaking the membrane and the use of sweeping gas on separation performance. Feed gas was used to soak the membrane module for 2 hours. Nitrogen gas was used as the sweeping gas. The results, shown in Tables 5-13, below, demonstrate that the carbon dioxide removal rate without soaking for example Table-5 (55%), Table-8 (51%), Table-11 (52%), whereas with soaking Table-6 (85%), Table-9 (80%), Table 12 (78%), and combination with soaking and sweeping gas Table-7 (94%), Table-10 (86%), Table-13 (83%).

TABLE 5
Without Soaking Feed/PO, PO or EO

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 57.72 | 15.89 | 40.77 |
| Total Mass Rate | kg/hr | 100 | 22.59 | 76.56 |
| Temperature | ° C. | 125 | 25.73 | 25.9 |
| Pressure | bar(g) | 22.01 | 21.51 | 0.5 |
| Total Molar Comp. | mol. % | | | |
| Percent | | | | |
| C₂H₄ | | 12.75 | 19.9 | 4.4 |
| O₂ | | 0.85 | 0.4 | 1.3 |
| CO₂ | | 72.86 | 61.0 | 86.7 |
| H₂O | | 2.16 | 3.4 | 0.7 |
| N₂ | | 0.83 | 0.9 | 0.8 |
| AR | | 2.14 | 2.6 | 1.6 |
| CH₄ | | 7.75 | 10.8 | 4.2 |
| C₂H₆ | | 0.65 | 1.0 | 0.2 |

[CO₂ Removal Rate = 55%]

TABLE 6
With Soaking in Feed/PO, PO or EO (Soaking time = 2 hours)

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 57.72 | 15.89 | 40.77 |
| Total Mass Rate | kg/hr | 100 | 22.59 | 76.56 |
| Temperature | ° C. | 125 | 25.73 | 25.9 |
| Pressure | bar(g) | 22.01 | 21.51 | 0.5 |
| Total Molar Comp. | mol. % | | | |
| Percent | | | | |
| C₂H₄ | | 12.75 | 36.53 | 3.81 |
| O₂ | | 0.85 | 0.48 | 1.02 |
| CO₂ | | 72.86 | 36.52 | 88.93 |
| H₂O | | 2.16 | 0 | 0.45 |
| N₂ | | 0.83 | 1.6 | 0.56 |
| AR | | 2.14 | 2.86 | 1.92 |
| CH₄ | | 7.75 | 20.11 | 3.14 |
| C₂H₆ | | 0.65 | 1.88 | 0.18 |

[CO₂ Removal Rate = 85%]

TABLE 7
With Soaking in Feed Gas/PO or EO (2 hours) + Sweeping
Gas (N₂) (similar to the feed flow rate on the tube side)

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 57.72 | 15.89 | 40.77 |
| Total Mass Rate | kg/hr | 100 | 22.59 | 76.56 |
| Temperature | ° C. | 125 | 25.73 | 25.9 |
| Pressure | bar(g) | 22.01 | 21.51 | 0.5 |
| Total Molar Comp. | mol. % | | | |
| Percent | | | | |
| C₂H₄ | | 12.75 | 45.73 | 2.03 |
| O₂ | | 0.85 | 1.21 | 0.73 |
| CO₂ | | 72.86 | 17.78 | 90.78 |
| H₂O | | 2.16 | 7.51 | 0.42 |
| N₂ | | 0.83 | 1.61 | 0.58 |
| AR | | 2.14 | 1.65 | 2.30 |
| CH₄ | | 7.75 | 22.42 | 2.98 |
| C₂H₆ | | 0.65 | 2.10 | 0.18 |

[CO₂ Removal Rate = 94%]

TABLE 8
Without Soaking in Benzene

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 13.17 | 37.53 | 3.95 |
| O₂ | | 0.87 | 0.51 | 1.01 |
| CO₂ | | 72.46 | 80.95 | 31.3 |
| H₂O | | 0.77 | 1.27 | 0.57 |
| N₂ | | 0.85 | 1.58 | 0.57 |
| AR | | 1.81 | 4.15 | 1.3 |
| CH₄ | | 7.81 | 20.1 | 3.17 |
| C₂H₆ | | 0.68 | 1.95 | 0.19 |

[CO₂ Removal Rate = 51%, Argon removal Rate = 45%]

TABLE 9
With Soaking in Benzene

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 13.15 | 37.54 | 3.91 |
| O₂ | | 0.87 | 0.51 | 1.01 |
| CO₂ | | 71.45 | 50.2 | 80.24 |
| H₂O | | 0.75 | 1.25 | 0.56 |
| N₂ | | 0.85 | 1.58 | 0.57 |
| AR | | 1.79 | 3.25 | 1.25 |
| CH₄ | | 7.81 | 20.1 | 3.17 |
| C₂H₆ | | 0.64 | 1.87 | 0.19 |

[CO₂ Removal Rate = 80%, Argon removal Rate = 50%]

TABLE 10
With Soaking in Benzene + Sweeping Gas

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 13.15 | 37.54 | 3.91 |
| O₂ | | 0.87 | 0.51 | 1.01 |
| CO₂ | | 71.55 | 40.25 | 95.15 |
| H₂O | | 0.75 | 1.25 | 0.56 |
| N₂ | | 0.85 | 1.58 | 0.57 |
| AR | | 1.81 | 2.29 | 1.64 |
| CH₄ | | 7.81 | 20.1 | 3.17 |
| C₂H₆ | | 0.64 | 1.87 | 0.19 |

[CO₂ Removal Rate = 86%, Argon removal Rate = 65%]

TABLE 11
Without Soaking in Methanol

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 12.75 | 36.53 | 3.81 |
| O₂ | | 0.85 | 0.48 | 1.02 |
| CO₂ | | 75.46 | 53.36 | 22.15 |
| H₂O | | 2.16 | 0 | 0.45 |
| N₂ | | 0.83 | 1.6 | 0.56 |
| Ar | | 2.16 | 3.275 | 0.99 |
| CH₄ | | 7.75 | 20.11 | 3.14 |
| C₂H₆ | | 0.65 | 1.88 | 0.18 |

[CO₂ Removal Rate = 52%, Argon removal Rate = 44%]

TABLE 12
With Soaking in Methanol

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 13.15 | 37.54 | 3.91 |
| O₂ | | 0.87 | 0.51 | 1.01 |
| CO₂ | | 73.46 | 49.15 | 67.05 |
| H₂O | | 0.75 | 1.25 | 0.56 |
| N₂ | | 0.85 | 1.58 | 0.57 |
| Ar | | 1.79 | 3.25 | 1.25 |
| CH₄ | | 7.81 | 20.1 | 3.17 |
| C₂H₆ | | 0.64 | 1.87 | 0.19 |

[CO₂ Removal Rate = 78%, Argon removal Rate = 52%]

TABLE 13
With Soaking in Methanol + Sweeping Gas

| | | Stream Name | | |
|---|---|---|---|---|
| | | FEED | RETENTATE | PERMEATE |
| | | Phase | | |
| | | vapor | vapor | vapor |
| Total Std. Vapor Rate | Nm³/hr | 58.14 | 16.01 | 41.77 |
| Total Mass Rate | kg/hr | 101 | 23.68 | 78.2 |
| Temperature | ° C. | 127 | 26.54 | 27.8 |
| Pressure | bar(g) | 22.5 | 21.2 | 0.7 |
| Total Molar Comp. | mol % | | | |
| Percents | | | | |
| C₂H₄ | | 12.75 | 36.53 | 3.81 |
| O₂ | | 0.85 | 0.48 | 1.02 |
| CO₂ | | 78.16 | 23.17 | 43.15 |
| H₂O | | 2.16 | 0 | 0.45 |
| N₂ | | 0.83 | 1.6 | 0.56 |
| Ar | | 2.21 | 4.65 | 3.79 |
| CH₄ | | 7.75 | 20.11 | 3.14 |
| C₂H₆ | | 0.65 | 1.88 | 0.18 |

[CO₂ Removal Rate = 83%, Argon removal Rate = 68%]

In the context of the present invention, at least the following 20 embodiments are disclosed. Embodiment 1 is a method of separating a gas mixture containing organic gaseous compounds and inorganic gaseous compounds. The method includes flowing the gas mixture into a membrane unit including a membrane element at least partially coated with an organic liquid. The method further includes separating the gas mixture in the membrane unit. The method still further includes flowing a first gas stream from the membrane unit, the first gas stream containing a lower concentration of inorganic gaseous compounds than the gas mixture. Embodiment 2 is the method of embodiment 1, wherein the gas mixture comprises ethylene, carbon dioxide, methane, and argon and the first gas stream includes a lower concentration of carbon dioxide and argon than the gas mixture. Embodiment 3 is the method of embodiment 2, wherein 90-93% of ethylene in the gas mixture is recovered. Embodiment 4 is the method of embodiment 2, wherein 85-87% CO₂ and 30-60% Ar is removed from the gas mixture. Embodiment 5 is the method of embodiment 2, wherein the gas mixture comprises 5 to 30 wt. % ethylene, 30 to 90 wt. % carbon dioxide, 1 to 10 wt. % argon, 1 to 10 wt. % methane, and 0.5 to 5 wt. % water. Embodiment 6 is the method of embodiment 2, wherein the first gas stream comprises 10 to 45 wt. % ethylene, 20 to 50 wt. % carbon dioxide, 0.5 to 8 wt. % argon, 5 to 20 wt. % methane, and 0.01 to 3 wt. % water. Embodiment 7 is the method of embodiment 2, wherein a second gas stream is flowed from the membrane unit, the second gas stream containing 0.1 to 4 wt. % ethylene, 70 to 98 wt. % carbon dioxide, 1 to 20 wt. % argon, 0.4 to 8 wt. % methane, and 0.1 to 1 wt. % water. Embodiment 8 is the method of embodiment 1, wherein the membrane element is at least partially coated with propylene oxide, ethylene oxide, methanol, benzene, or combinations thereof. Embodiment 9 is the method of embodiment 8, wherein the method further includes soaking the membrane in propylene oxide, ethylene oxide, methanol, benzene, or combinations thereof for 2 to 5 hours before the flowing of the gas mixture into the membrane unit. Embodiment 10 is the method of embodiment 1, wherein the membrane unit includes a membrane element that comprises per-fluoro-polymer and/or cellulose acetate. Embodiment 11 is the method of embodiment 1, wherein the gas mixture includes a reclaim gas stream from an ethylene oxide-glycol production plant. Embodiment 12 is the method of embodiment 1 further including flowing nitrogen at 20 to 30% of mass flow into the membrane unit as a sweeping gas. Embodiment 13 is the method of embodiment 1 further including flowing nitrogen at 20 to 30% of mass flow into the membrane unit as a sweeping gas.

Embodiment 14 is a system for separating a gas mixture containing organic gaseous compounds and inorganic gaseous compounds. The system includes a membrane unit containing a membrane element at least partially coated with an organic liquid. Embodiment 15 is the system of embodiment 14, wherein the membrane element includes per-fluoro-polymer and/or cellulose acetate. Embodiment 16 is the system of embodiment 14, wherein the membrane element is at least partially coated with propylene oxide, ethylene oxide, methanol, benzene, or combinations thereof. Embodiment 17 is the system of embodiment 14, wherein the gas mixture comprises ethylene, carbon dioxide, methane and argon. Embodiment 18 is the system of embodiment 17, wherein the membrane unit is adapted to separate the gas mixture to produce a first gas stream that includes a lower concentration of carbon dioxide and argon than the gas mixture. Embodiment 19 is the system of embodiment 18, wherein the membrane unit is adapted to separate the gas mixture to produce a second gas stream that includes a higher concentration of carbon dioxide and argon than the gas mixture. Embodiment 20 is the system of embodiment 14, further including a compressor for flowing a sweeping gas through the membrane unit.

All embodiments described above and herein can be combined in any manner unless expressly excluded.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of separating a gas mixture (100) comprising ethylene, carbon dioxide, methane, and argon, the method comprising:
flowing the gas mixture (100) into a membrane unit (101) comprising a membrane element (102) at least partially coated with an organic liquid;
separating the gas mixture (100) in the membrane unit (101); and
flowing a first gas stream (104) from the membrane unit (101), the first gas stream (104) comprising a lower concentration of carbon dioxide and argon than the gas mixture (100).

2. The method of claim 1, wherein the membrane unit (101) operates at a temperature in the range of 80° C. to 200° C.; wherein the membrane unit (101) operates at a pressure in the range of 10 bar to 50 bar; and wherein the membrane unit (101) operates at a flow rate of 200 to 30000 kg/hour.

3. The method of claim 1, wherein 90-93% of ethylene in the gas mixture (100) is recovered.

4. The method of claim 1, wherein 85-87% CO₂ and 30-60% Ar is removed from the gas mixture (100).

5. The method of claim 1, wherein the gas mixture (100) comprises 5 to 30 wt. % ethylene, 30 to 90 wt. % carbon dioxide, 1 to 10 wt. % argon, 1 to 10 wt. % methane, and 0.5 to 5 wt. % water.

6. The method of claim 1, wherein the first gas stream (104) comprises 10 to 45 wt. % ethylene, 20 to 50 wt. % carbon dioxide, 0.5 to 8 wt. % argon, 5 to 20 wt. % methane, and 0.01 to 3 wt. % water; wherein a second gas stream (103) is flowed from the membrane unit (101), the second gas stream (103) comprising 0.1 to 4 wt. % ethylene, 70 to 98 wt. % carbon dioxide, 1 to 20 wt. % argon, 0.4 to 8 wt. % methane, and 0.1 to 1 wt. % water.

7. The method of claim 1, wherein the organic liquid is selected from the group comprising of propylene oxide, ethylene oxide, methanol, benzene, or combinations thereof.

8. The method of claim 1, wherein the membrane element (102) is at least partially coated with propylene oxide, ethylene oxide, methanol, benzene or combinations thereof.

9. The method of claim 1, wherein the method further comprises:
soaking the membrane in propylene oxide, ethylene oxide, methanol, benzene, or combinations thereof for 2 to 5 hours before the flowing of the gas mixture (100) into the membrane unit (101).

10. The method of claim 1, wherein the membrane unit (101) comprises a membrane element (102) that comprises per-fluoro-polymer and/or cellulose acetate

11. The method of claim 1 further comprising:
flowing nitrogen at 20 to 30% of mass flow into the membrane unit (101) as a sweeping gas.

12. A system (10) for separating a gas mixture (100) comprising ethylene, carbon dioxide, methane, and argon, the system comprising:
a membrane unit (101) comprising a membrane element (102) at least partially coated with an organic liquid.

13. The system of claim 13, wherein the membrane element (102) comprises per-fluoro-polymer and/or cellulose acetate.

14. The system of claim 13, wherein the membrane element (102) is at least partially coated with propylene oxide, ethylene oxide, methanol, benzene, or combinations thereof.

15. The system of claim 13, further comprising a compressor for flowing a sweeping gas through the membrane unit.
